(19) United States
(12) Patent Application Publication
(10) Pub. No.: US 2020/0222675 A1
(43) Pub. Date: Jul. 16, 2020

(54) SINUS DILATION

(71) Applicant: MEDTRONIC XOMED, INC., Jacksonville, FL (US)

(72) Inventor: James Britton Hissong, Jacksonville, FL (US)

(73) Assignee: MEDTRONIC XOMED, INC., Jacksonville, FL (US)

(21) Appl. No.: 16/739,835

(22) Filed: Jan. 10, 2020

Related U.S. Application Data
(60) Provisional application No. 62/791,075, filed on Jan. 11, 2019.

(51) Int. Cl. A61M 29/00

(52) U.S. Cl. CPC A61M 29/00; A61M 2210/0681

(57) ABSTRACT

A sinus dilation instrument including a handle, an inner member, a balloon and an outer member. The inner member defines a proximal portion opposite a distal portion. The proximal portion is coupled to the handle. The distal portion terminates at a distal end opposite the handle. The balloon fixedly is coupled to an exterior of the inner member along the distal portion. The outer member is slidably disposed over the inner member, and includes a first section and a second section. The second section is more rigid than the first section. The first section terminates at a terminal end opposite the second section. The outer member is transitionable relative to the inner member between an advanced position and a retracted position, the advanced position including the balloon disposed within the first section and the retracted position including the balloon located distal the terminal end.

### CROSS REFERENCE TO RELATED APPLICATION

This Non-Provisional Utility application claims the benefit of the filing date of U.S. Provisional Patent Application Ser. No. 62/791,075, filed Jan. 11, 2019, titled “SINUS DILATION,” the entire teachings of which are incorporated herein by reference.

### FIELD

The present technology is generally related to sinus dilation systems and methods. More particularly, it relates to minimally invasive, balloon-based systems and methods for dilating a portion of a patient's paranasal sinuses in the treatment of sinusitis and other disorders.

### BACKGROUND

The paranasal sinus system is a grouping of four pairs of air-filled cavities that are named for the facial bones in which they are located. The maxillary sinuses surround the nasal cavity, the frontal sinuses are above the eyes, the ethmoid sinuses are between the eyes, and the sphenoid sinuses are within the sphenoid bone at the center of the skull base under the pituitary gland. The paranasal sinuses are lined with respiratory epithelium, are joined to the nasal cavity via small orifices called ostia, and contain secretory tissue that produces a large volume of mucus. This mucus is normally relieved from the sinuses in a specific pattern through the corresponding ostia.

The mucus membrane that lines the paranasal sinuses can become inflamed. This inflammation is known as sinusitis (or rhinosinusitis), and can be caused by various factors such as bacteria, viruses, allergies, anatomical abnormalities, etc. If the mucosa of one of the paranasal sinus passageways becomes inflamed, the passageway can become blocked, trapping mucus. Patients suffering from sinusitis can experience a number of symptoms or complications, such as headache, facial pain, toothache, inner ear problems, etc. Widening the walls of the sinus passageway, with the goal of restoring normal drainage without damaging the sinus lining, can be useful to alleviate the patient's symptoms. Sinus dilation devices including balloons can be used to expand the ostium (opening pathway) into three sinus cavities including the maxillary, sphenoid and frontal.

### SUMMARY

The techniques of this disclosure generally relate to sinus dilation systems and methods including a balloon dilation device.

In one aspect, the present disclosure provides surgical dilation instrument includes an inner member, a balloon, an outer member, and a handle. The inner member includes a proximal portion and a distal portion. The balloon is disposed around the inner member at the distal portion. A distal end of the balloon is fixedly coupled to the inner member at the distal portion. The balloon has an inflated state and a deflated state. The outer member has a first section and a second section. The first section is comprised of a flexible material and the second section is comprised of a rigid material. The outer member is slidably disposed around the inner member with the first section slidably disposable over the balloon when the balloon is in the deflated state. The handle includes an actuator. The actuator is coupled to the second section of the outer member. The second section is configured to rigidly transfer movement of the actuator to the first section to slidably move the outer member with respect to the inner member and the balloon while the inner member and balloon are longitudinally fixed relative to the handle.

In another aspect, the disclosure provides method of dilating a sinus cavity including inserting a dilation device into a sinus cavity. The dilation device including an inner member, a balloon, and an outer member. The balloon fixedly is coupled to a distal portion of the inner member. The outer member is an elongated tubular member including a flexible distal section and a rigid proximal section. The flexible distal section of the outer member is slidably disposed over the balloon. The method includes retracting the outer member from the balloon and expanding the balloon. The method includes treating a site of the sinus cavity with the expansion of the balloon. The method includes deflating the balloon and slidably moving the flexible distal section of the outer member over the balloon and then withdrawing the dilation device from the sinus cavity.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a perspective view that illustrates a sinus dilation system in accordance with aspects of the present disclosure.

FIG. 2A is a cross-sectional view that illustrates an example sinus dilation instrument in an inflated state in accordance with aspects of the present disclosure.

FIG. 2B is a cross-sectional view that illustrates an example sinus dilation instrument in a deflated state in accordance with aspects of the present disclosure.

FIGS. 3A-3F are diagrammatic side views of a sinus dilation instrument in states of use in accordance with aspects of the present disclosure.

### DETAILED DESCRIPTION

Surgical devices and systems embodying principles of the present disclosure can be employed in various types of surgical procedures including, but not limited to, treatment of sinusitis and Eustachian tube dysfunction. The sinus ostiums are small and the space in the nasal passages and sinus airways is limited. Sinus dilation balloons used to expand the ostiums can become damaged by contact with exposed bone, cartilage, or another tool while moving the balloon through the sinus passageways during the sinus dilation procedure. If the balloon is damaged, it may not inflate or may burst pre-maturely when being inflated to the high pressures needed to break bone and cartilage under the mucosal surface to expand the ostiums. The surgical treatment devices and systems, in accordance with aspects of the present disclosure, can provide for ease of use to the surgeon by allowing the surgeon to operate a balloon dilation device including insertion and withdrawal of the balloon dilation device in one or more sinuses or other cavities of the patient without damaging the balloon and without manually manipulating the balloon on the device to prepare the balloon dilation device for insertion or re-insertion into the patient. Additionally, the surgical treatment devices and systems, in accordance with aspects of the present disclosure, can provide ease of insertion of a dilating balloon and can enhance the ease of positioning the dilating balloon by providing increased visibility of the anatomy and physiology of the tissues as well as for navigating through the tissues, such as during insertion.

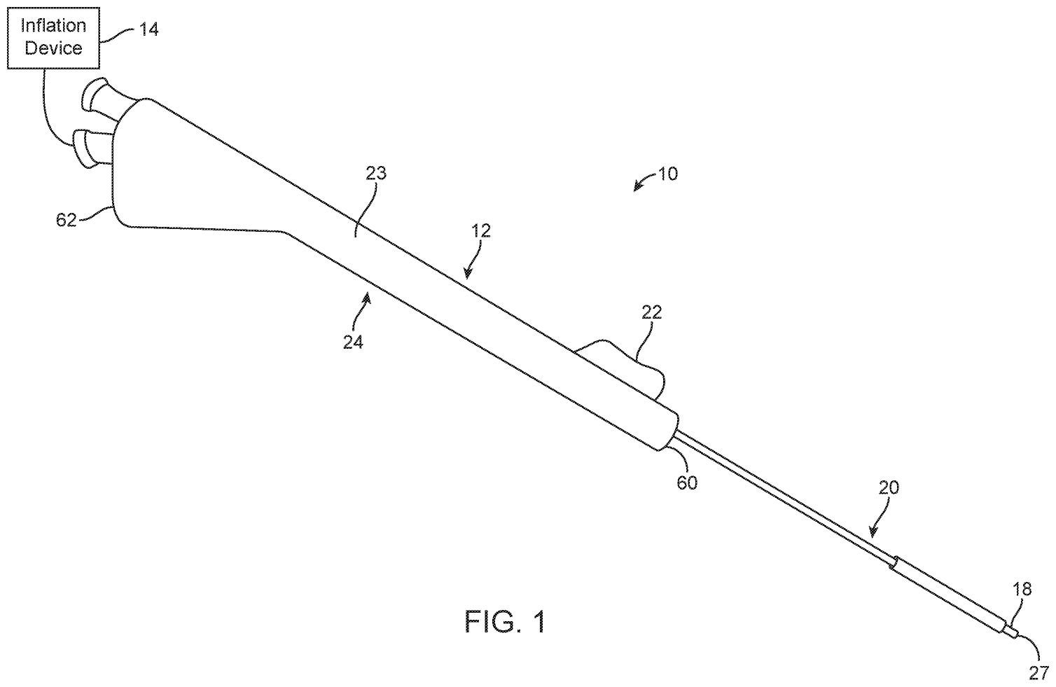

One embodiment of a surgical dilation system 10 in accordance with principles of the present disclosure is illustrated in FIG. 1. The surgical dilation system 10 includes a dilation instrument, or dilation device, 12 and an inflation device 14. The sinus dilation instrument 12 includes an inner member 16 (hidden in the view of FIG. 1), a balloon 18, and an outer member 20. The inflation device 14 is selectively fluidly connected to the instrument 12 at a handle 24, and operates to effectuate inflation and deflation of the balloon 18. In general terms, the balloon 18 is fixedly attached to the inner member 16, and the outer member 20 is slidable to extend over the inner member 16 and the balloon 18. The outer member 20 is illustrated as extended over balloon 18 in FIG. 1. The components can be carried by the handle 24 and are described in greater detail below. The handle 24 can include an actuator 22 (e.g., button) carried by a base 23; the actuator 22 can be manipulated by a user relative to the base 23 to slidably move the outer member 20 longitudinally along the inner member 16 to selectively extend the outer member 20 over the balloon 18. As identified in FIG. 1, the handle 24 can be viewed as defining a distal end 60 opposite a proximal end 62. The instrument 12 can be sized and shaped for positioning the balloon 18 carried by the instrument 12 at a particular targeted sinus region (e.g., frontal sinus, maxillary sinus, or sphenoid sinus) via a patient's naris (or alternatively sized and shaped for accessing the targeted sinus region through other conventional approaches such as canine fossa or open approach).

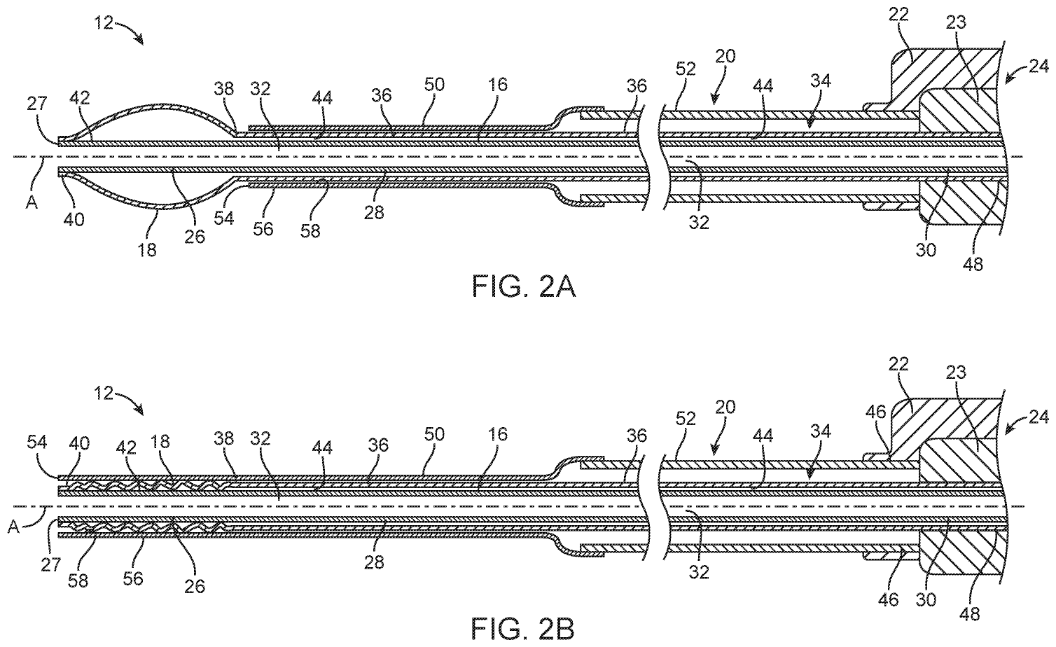

FIGS. 2A and 2B illustrate enlarged partial cross-sectional views of an example surgical dilation instrument 12 in accordance with aspects of the present disclosure. As a point of reference, FIG. 2A illustrates the outer member 20 withdrawn or retracted from the balloon 18, with the balloon 18 in the inflated or expanded state. FIG. 2B illustrates the outer member 20 extended over the balloon 18, with the balloon 18 in a deflated state.

The inner member 16 is an elongated body extending along a longitudinal axis “A” and defining a distal portion 26 terminating at a distal tip 27, an intermediate portion 28, and a proximal portion 30. The proximal portion 30 of the inner member 16 is coupled to the handle 24. Although illustrated as being straight and extending linearly, it is understood that the inner member 16 can include curves, bends, etc. The inner member 16 can be malleable, to be bent into the desired shape by a surgeon prior to insertion into a patient. In some embodiments, the distal portion 26 of the inner member 16 is pre-bent. The intermediate portion 28, as well as the distal portion 26, can be configured for accessing the frontal or other sinus via the naris, for example. The inner member 16 can be formed of a malleable surgically safe material, such as stainless steel or surgical grade aluminum, for example. The inner member 16 can have a round, oval, or other appropriate cross-sectional shape. Although illustrated as tubular, the inner member 16 can be tubular (i.e., hollow) or solid. In some embodiments, the inner member 16 defines a lumen 32 extending along a length of the inner member 16 between the tip 27 and a proximal end (not shown) that is suitable for a guidewire and/or a tracking device to be extended within.

In some embodiments, and as best shown in FIGS. 2A-2B, the balloon 18 is provided or formed as part of an inflatable sheath 34. The sheath 34 can be a homogeneous, extruded tubular body that defines the balloon 18 and a trailing section 36. The sheath 34 can be comprised of a semi-compliant material that is non-stretchable and high strength. The sheath 34 can be a polymeric material (e.g., nylon, nylon derivatives, Pebax, polyurethane, PET, etc.). The trailing section 36 extends proximally from a proximal end 38 of the balloon 18, and is generally sized and shaped in accordance with a size and shape of the inner member 16. The balloon 18 can be defined along a length of the sheath 34 in various manners, and is generally characterized as being expandable whereas the trailing section 36 is generally characterized as being non-expandable (e.g., a hoop strength of the tailing section 36 is greater than a hoop strength of the balloon 18). In some embodiments, the balloon 18 has a length between the proximal end 38 and a distal end 40 of 16 to 24 millimeters (mm).

With the above constructions, the distal end 40 of the balloon 18 is sized and shaped to receive, and be coupled to, the distal tip 27 or the distal portion 26 of the inner member 16. The distal end 40 of the balloon 18 is directly bonded to an exterior surface 42 of the inner member 16. The proximal end 38 of the balloon 18 is not bonded to the exterior surface 42 of the inner member 16, allowing fluid to flow through an inflation path 44 to enter, or exit, the balloon 18 during inflation or deflation. The balloon 18 expands to, but not beyond, a preformed size and shape reflected in FIG. 2A at the expected operational inflation pressures. The balloon 18 in the deflated (or contracted) state shown in FIG. 2B is loosely formed over the inner member 16. In some embodiments, the balloon 18 is configured to have a maximum outer diameter upon inflation of about 7 mm and a circumference of 22 mm and in an uninflated, or deflated state, the balloon 18 can have evacuated lay-flat width of approximately 11 mm; in other embodiments, the balloon is configured to have a maximum outer diameter upon inflation of about 17 mm and a circumference of 53 mm and in an uninflated, or deflated state, the balloon 18 can have evacuated lay-flat width of approximately 25 mm.

The balloon 18 is secured over and fixedly attached to the inner member 16. The balloon 28 consistently expands or inflates to the predetermined shape at the distal portion 26 of the inner member 16. As a point of reference, the sheath 34 is shown with the balloon 18 in the expanded state in FIG. 2A and centered circumferentially around the inner member 16. However, the sheath 34 as a standalone component need not have a definitive shape or position with respect to the inner member 16, but instead is sufficiently flexible to generally follow or conform to a shape or curvature of the inner member 16 upon final assembly. The balloon 18 and the trailing section 36 are tubular, and can be separately formed and subsequently assembled in completing the sheath 34. In one embodiment, the trailing section 36 can have an increased wall thickness to that of the balloon 18. The trailing section 36 can experience minimal, if any, expansion when the sheath 34 is subjected to expected operational inflation pressures useful for inflating, or expanding, the balloon 18.

The inner member 16 can be an elongated probe mounted to the handle 24. The handle 24 and the inner member 16 can be formed separately and subsequently assembled to one another. The inner member 16 can extend within a passageway 48 of the handle 24. The handle 40 can assume a variety of forms and in some embodiments is formed of a hardened, surgically safe material such as plastic or metal. While the handle 24, and in particular the base 23, can have the generally cylindrical, streamlined shape shown, any other shape conducive to grasping and manipulating by a user's hand is equally acceptable. The handle 24 can incorporate various features such as the actuator 22 configured to interface with or retain the outer member 20 of the dilation instrument 12. In some embodiments, the handle 24 is constructed to provide access to the inflation lumen 32 of the sheath 34. For example, the handle 24 can fluidly connect the inflation device 14 (see, e.g., FIG. 1) to the inflation lumen 32. In some embodiments, the inner member 16 is fixedly coupled or rigidly affixed relative to the base 23. Similarly, the sheath 34 can be fixedly coupled or rigidly affixed relative to the base 23.

The outer member 20 can be mounted to the handle 24 in a variety of manners (insert molded, adhesive, welded, press fit, etc.), with the outer member 20 extending distally from the handle 24. For example, the handle 24 can be press fit over the outer member 20 such that a proximal end 46 of the outer member 20 is encompassed within or coupled to the actuator 22. The outer member 20 is curved or bent to follow the curved or bent shape of the inner member 16 that is coaxially disposed over.

The outer member 20 includes a first section 50 and a second section 52. The second section 52 connects to, and extends from, the handle 24 and the actuator 22 of the handle 24. The first section 50 extends from the second section 52 to terminate at a distal end 54 of the outer member 20. The second section 52 is formed of a rigid material, such as stainless steel, for example. Other suitable rigid materials are also acceptable. The second section 52 can rigidly transfer movement of the actuator 22 to the first section 50. The first section 50 is selectively deployable over the balloon 18 with the outer member 20 slidably movable along the exterior of the sheath 34 and the inner member 16 that the balloon 18 is fixedly disposed upon. In other words, the inner member 16 and the balloon 18 attached to the inner member 16 are longitudinally fixed relative to the base 23, and the outer member 20 is slidably disposed around the inner member 16 and balloon 18 to be selectively extendable over the balloon 18 by a user manipulating the actuator 22 on the handle 24. The distal end 54 of the outer member 20, and more particularly, of the first section 50, can include a terminal end that is inwardly tapered from an outer wall surface 56 to an inner wall surface 58. For example, the distal end 54 can be angled, or tapered, at a 30 degree angle. Other suitable angles are also acceptable. The tapering of the distal end 54 can aid in facilitating movement of the first section 54 over the balloon 18.

The outer member 20 in an extended position around, or over, the balloon 18 in the deflated state, minimizes an outer profile of the instrument 12 along the balloon 18. In some embodiments, the outer member 20 over the deflated balloon 18 provides an outer diameter on the order of 3 mm in the deflated or uninflated state. The first section 50 can capture and aid in the collapse the deflated balloon 18. The first section 50 can be thin-walled, having a wall thickness on the order of 0.25 mm-0.5 mm; in other embodiments a wall thickness of approximately 0.10 mm. In one embodiment, the first section 50 is formed of a polymeric material, such as polytetrafluoroethylene (PTFE), although other suitable materials are also acceptable. The first section 50 can have a length suitable to fully extend over the length of the balloon 18. In one example, when the balloon 18 has a length between the distal and proximal ends 38, 40 of 17 mm, the first section 50 can have a length 18-20 mm.

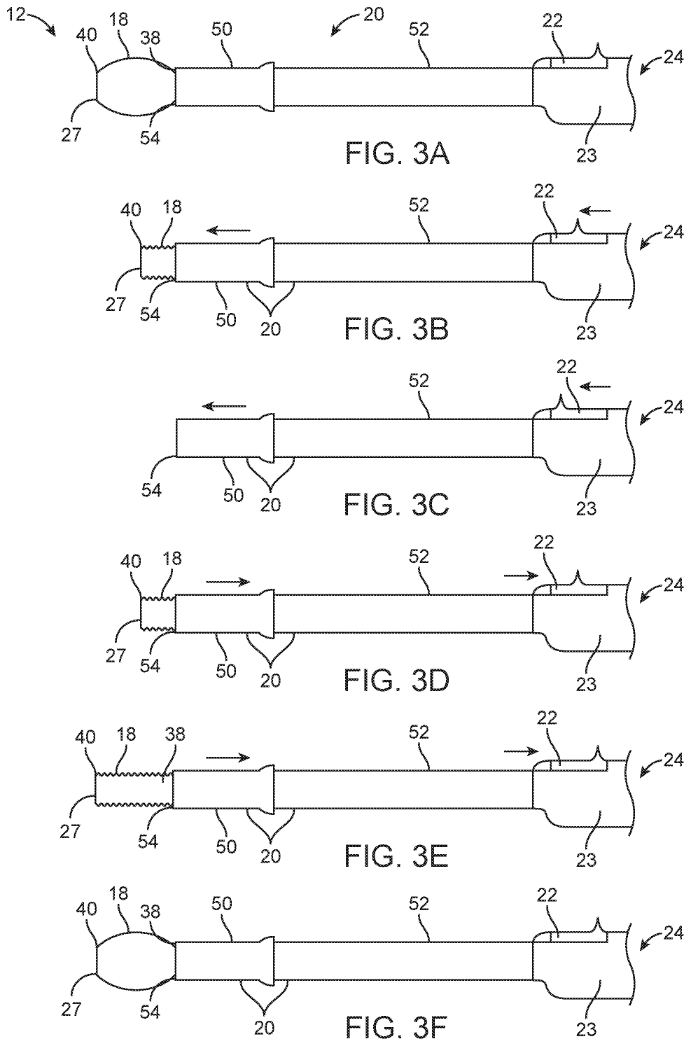

FIGS. 3A-3D are diagrammatic side views of a sinus dilation instrument in states of use in accordance with aspects of the present disclosure. FIG. 3A illustrates the balloon 18 in an inflated state with the first section 50 of the outer member 20 retracted, or withdrawn, from the balloon 18 fixedly disposed on the inner member 16 (not shown). The distal end 54 of the outer member 20 can be proximal, or adjacent to, the proximal end 38 of the balloon. In FIG. 3B, the balloon 18 has been deflated and a user selectively manipulates the actuator 22 relative to the base 23, effectively toward the distal end 60 the handle 24 to move the outer member 20 over the balloon 18 and toward the distal end 27 of the inner member 16. The manipulation of the actuator 22 transfers movement to the rigid second section 52, which in turn, transfers longitudinal movement to the flexible first section 50. The proximal end 38 of the balloon 18 is encapsulated, or covered, by the first section 50 as the first section 50 is moved toward the distal end 27 of the inner member 16. The inner member 16, and the balloon 18 attached to the inner member 16, remain longitudinally fixed with respect to the base 23 of the handle 24. FIG. 3C illustrates the first section 50 moved distally until fully extended over the balloon 18 to cover and protect the balloon 18 and minimize the outer profile of the dilation device 12. The dilation device 12 is ready for insertion into the patient at this state. FIG. 3D illustrates the actuator 22 manipulated or retracted relative to the base 23, in a direction toward the proximal end 62 (FIG. 1) of the handle 24 to move the outer member 20 proximally and begin releasing the balloon 18 from the first section 50. FIG. 3E illustrates the outer member 20 moved proximally to fully release, or expose, the balloon 18 in the deflated state for inflation of the balloon 18. FIG. 3F illustrates the balloon 18 in an inflated state.

With this construction, the system 10 is useable in treating the paranasal sinus system. In general terms, sinus dilation device 12 useful for treating sinusitis employs a small, flexible balloon 18 to enlarge the affected sinus passageway(s). Once the surgeon has determined the paranasal sinus to be treated, the surgeon shapes sinus dilation instrument into the desired shape. The inflation device 14 is operated to inflate the balloon 18, thereby expanding the sinus ostium (or other region of the accessed sinus) as desired. When the balloon 18 is correctly located, the balloon 18 is inflated to widen the walls of the sinus passageway, with the goal of restoring normal drainage without damaging the sinus lining. When performing sinus dilation, the surgeon can insert the sinus dilation device 12 through the nostril (or naris) to gain access to the affected sinus ostia (opening) under endoscopic visualization. Once access to the intended targeted location is confirmed, the sinus dilation device 12, carrying the balloon 18, can be introduced into the sinus cavity, locating the balloon in the blocked ostium. Once the desired position of the balloon 18 has been visually confirmed, the balloon 18 can be gradually inflated to dilate the narrowed or blocked ostium. Following deflation of the balloon 18, the sinus dilation device 12 is removed from the patient and the procedure is complete. The balloon is then deflated and the sleeve slid over the balloon for removal from the sinus cavity, and, if desired, inserted into another sinus cavity. Following deflation of the balloon 18, the first section 50 of the outer member 20 is slid over the balloon 18 and the sinus dilation device 12 is removed from the patient. The outer member 20 protects the balloon 18 from damage from instruments and sharp surfaces within the sinuses when the balloon 18 is deflated (e.g., during insertion and withdrawal) and improves the surgeon's visibility around the sinus dilation device 12 with containment of the deflated balloon 18.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

What is claimed is:

1. A sinus dilation instrument for use in dilating a region of a patient's nasal sinus system, the sinus dilation instrument comprising:
a handle;
an inner member defining a proximal portion opposite a distal portion, wherein the proximal portion is coupled to the handle, and further wherein the distal portion terminates at a distal end opposite the handle;
a balloon fixedly coupled to an exterior of the inner member along the distal portion;
an outer member slidably disposed over the inner member, the outer member including a first section and a second section, the second section being more rigid than the first section, wherein the first section terminates at a terminal end opposite the second section;
wherein the outer member is transitionable relative to the inner member between an advanced position and a retracted position, the advanced position including the balloon disposed within the first section and the retracted position including the balloon located distal the terminal end.

2. The sinus dilation instrument of claim 1, wherein the handle includes an actuator slidably connected to a base, and further wherein the inner member is fixedly coupled to the base, and even further wherein the second section is fixedly coupled to the actuator.

3. The sinus dilation instrument of claim 1, wherein the first section is formed of a polymer material and the second section is formed of a metal.

4. The sinus dilation instrument of claim 3, wherein the metal is stainless steel.

5. The sinus dilation instrument of claim 1, wherein the balloon is defined by a sheath, and further wherein a trailing section of the sheath extends from the balloon to the handle.

6. The sinus dilation instrument of claim 5, wherein an inflation path to an interior of the balloon is defined between an inner surface of the sheath and the exterior surface of the inner member.

7. The sinus dilation instrument of claim 5, wherein a hoop strength of the trailing section is greater than a hoop strength of the balloon.

8. The sinus dilation instrument of claim 7, wherein a proximal end of the balloon is defined at a point of transition to the trailing section, and further wherein the proximal end of the balloon is free of attachment to the exterior surface of the inner member.

9. The sinus dilation instrument of claim 1, wherein the instrument is configured to direct the balloon, in a deflated state, through a naris of the patient in response to manual manipulation of the handle.

10. A method of dilating a region of a patient's nasal sinus system, the method comprising:
receiving a sinus dilation instrument configured for accessing the region of the patient's nasal sinus system, the sinus dilation instrument including:
a handle,
an inner member defining a proximal portion opposite a distal portion, wherein the proximal portion is coupled to the handle, and further wherein the distal portion terminates at a distal end opposite the handle,
a balloon fixedly coupled to an exterior of the inner member along the distal portion,
an outer member slidably disposed over the inner member, the outer member including a first section and a second section, the second section being more rigid than the first section, wherein the first section terminates at a terminal end opposite the second section,
wherein the outer member is transitionable relative to the inner member between an advanced position and a retracted position, the advanced position including the balloon disposed within the first section and the retracted position including the balloon located distal the terminal end;
with the balloon in a deflated state, inserting a distal segment of the sinus dilation instrument through a naris of the patient to locate the balloon at a target site;
inflating the balloon to dilate the target site;
deflating the balloon after dilating the target site;
advancing the first section over the deflated balloon; and
removing the sinus dilation instrument from the patient.

11. The method of claim 10, wherein prior to the step of inserting a distal segment of the sinus dilation instrument through a naris of the patient, the method further comprising:
advancing the first section over the deflated balloon;
retracting the first section relative to the balloon to expose the balloon;
wherein the step of inflating follows the step of retracting.

12. The method of claim 10, wherein the handle includes an actuator slidably connected to a base, and wherein the inner member is fixedly coupled to the base, and wherein the second section is fixedly coupled to the actuator, and even further wherein the step of advancing includes sliding the actuator relative to the base.

13. The method of claim 12, wherein the step of advancing includes the second section transferring a sliding force from the actuator to the first section.

14. The method of claim 10, wherein an interior of the balloon is fluidly connected to an inflation pathway extending to the handle.

15. The method of claim 14, wherein the step of inflating includes directing an inflation medium through the inflation pathway to the balloon.

16. The method of claim 10, wherein the target site is a nasal sinus ostium.

17. A system for dilating a region of a patient's nasal sinus system, the system comprising:
a sinus dilation instrument configured for accessing the region of the patient's nasal sinus system, the sinus dilation instrument including:
a handle,
an inner member defining a proximal portion opposite a distal portion, wherein the proximal portion is coupled to the handle, and further wherein the distal portion terminates at a distal end opposite the handle,
a balloon fixedly coupled to an exterior of the inner member along the distal portion,
an outer member slidably disposed over the inner member, the outer member including a first section and a second section, the second section being more rigid than the first section, wherein the first section terminates at a terminal end opposite the second section,
wherein the outer member is transitionable relative to the inner member between an advanced position and a retracted position, the advanced position including the balloon disposed within the first section and the retracted position including the balloon located distal the terminal end;
an inflation device fluidly connected to the sinus dilation instrument.

18. The system of claim 17, wherein the sinus dilation system defines an inflation pathway from an interior of the balloon to the handle, and further wherein the inflation device is fluidly connected to the inflation pathway.

19. The system of claim 17, wherein the handle includes an actuator slidably connected to a base, and further wherein the inner member is fixedly coupled to the base, and even further wherein the second section is fixedly coupled to the actuator.

20. The system of claim 17, wherein the first section is formed of a polymer material and the second section is formed of a metal.
